(19) United States
(12) Patent Application Publication
(10) Pub. No.: US 2018/0317959 A1
(43) Pub. Date: Nov. 8, 2018

(54) METHOD AND DEVICE FOR PRODUCING CANNULAS

(71) Applicant: RENA GmbH, Guetenbach (DE)

(72) Inventors: Philipp Bauer, Furtwangen (DE); Sascha Kuner, Furtwangen (DE); Werner Saule, Voerstetten (DE); Frank Schienle, Freiburg (DE); Martin Weber, Biederbach (DE); Robert Zschaler, Schoenwald (DE)

(21) Appl. No.: 15/525,003

(22) PCT Filed: Nov. 5, 2015
(86) PCT No.: PCT/EP2015/075804

§ 371 Date: May 5, 2017

(30) Foreign Application Priority Data
Nov. 7, 2014 (DE) 102014116287.0

(51) Int. Cl. A61B 17/34; B23H 9/08; B23H 3/00; B23H 1/00

(52) U.S. Cl. CPC A61B 17/3421; B23H 9/08; A61B 2017/00526; B23H 1/00; A61B 2017/3456; B23H 3/00

(57) ABSTRACT

The present invention relates to a method for the manufacture of cannulae, as well as cannulae that have been manufactured using the method. The invention further relates to a device that is suitable for the manufacture of cannulae using the method.

The method involves the manufacture of a cannula from a hollow tubular segment (1), with a penetrating entity that has a leading penetrating section and a trailing aperture section, wherein the leading penetrating section has a beveled surface that extends rearward from a penetrating tip and has inner lateral edges, and wherein the trailing aperture section has a beveled surface that extends rearward from the beveled surface of the leading section and has inner lateral edges as well as a rounded trailing edge (10, 12), wherein the rounding of trailing edge (10, 12) is produced by means of electrochemical machining (ECM) in the presence of an electrolyte.

### CROSS REFERENCE TO RELATED APPLICATIONS

This is a US national phase application of international patent application no. PCT/EP2015/075804, filed Nov. 5, 2015, which itself claims priority to German patent application DE 10 2014 116 287.0, filed Nov. 7, 2014. Each of the applications referred to in this paragraph are herein incorporated by reference in their entireties.

### TECHNICAL FIELD

The invention relates to a method for the manufacture of cannulae, as well as to cannulae that have been manufactured using the method. The invention further relates to a device that is suitable for the manufacture of cannulae using the method.

### BACKGROUND OF THE INVENTION

Cannulae normally have a sharp puncture tip as well as exterior cutting edges, but can also be designed without a sharp tip and without exterior cutting edges, as is the case with epidural cannulae, for example. The trailing end of the cannula aperture, which is also frequently described as the heel end or eye, is usually elliptical. Because the inner lateral edges and the trailing elliptical edge of the eye are sharp, medical cannulae in particular, which are used to puncture skin or other materials, have the consistent tendency to punch out particles from the skin or from other materials.

The punching tendency can be reduced through numerous techniques known in the prior art, such as e.g. glass bead blasting and fine sand blasting, as well as special cannula cuts.

DE-OS 26 00 299 discloses a cannula as well as a method and a device for its manufacture. The cannulae are acquired by means of a typical bias cut from a tubular segment and have rounded inner edges in the trailing area of the cannula aperture (penetrating edge), wherein these rounded edges are created through the use of brushes with abrasive properties. For this purpose, the device comprises a rotating brush with multiple bristles furnished with an abrasive means, or a brush onto which abrasive material is introduced, as well as a clamping plate for detachably fastening a plurality of cannula to a supporting plate as an accommodating surface. The bristles process the apertures of multiple cannulae consecutively in order to round off and blunt the inner lateral edges in the trailing area of the cannula apertures and the trailing edges (eyes) thereof. Thus, a limited brushing of the cannula apertures occurs only in the area of the penetrating edges. The disadvantage of this solution, however, is that it is impossible to rotate the cannulae within the device in order to enable, in addition to the bias cut, also a lateral grinding on both sides in the leading section of the cannula aperture through so-called facet cuts. Another significant disadvantage of the proposed method is evident from the use of grinding discs and brushes in the mechanical handling steps. The interior of the cannulae are thereby contaminated with grinding debris and are also subject to mechanical stress due to direct contact with the grinding disc or the bristles of the brush, which makes additional processing steps necessary.

In the DE-OS 32 30 735 A1, a method for the manufacture of cannulae through segmented cutting of cannula tubes is described, wherein the cutting is performed by means of electrical discharge machining. The device proposed for the method enables an automated cutting of a plurality of cannula tubes, wherein the cannula tubes, which are provided in bundles, are not rotated relative to their longitudinal axis during the electrical discharge (electroerosion) cutting, since the subsequently required creation of a cannula tip is achieved through the use of common grinding methods, for which reason the aforementioned disadvantages of mechanical treatment also exist here.

Patent specification DE 103 27 067 B4 likewise describes a method for the simultaneous manufacture of multiple cannulae, which are held on a jig in parallel and at a distance from one another. The cannulae are beveled sequentially or simultaneously through grinding and/or erosion. It proposes deburring the beveling through fine sand blasting or glass bead blasting, wherein contamination of the cannula occurs, which must be removed with additional effort in subsequent process steps. A processing (rounding) of the trailing end of the cannula aperture (heel end, eye) is not addressed in this publication.

Patent specification DE 10 2011 112 021 B4 describes the manufacture of a minimal-puncture cannula using a new type of cannula cut. It states that the punching problem is solved through the use of the cutting technology being presented, which leads to a special tip geometry, for which reason a separate or integrated processing of the trailing end of the cannula aperture for rounding or deburring thereof is not addressed.

### SUMMARY OF THE INVENTION

In accordance with the above, the object of the present invention is to provide a method and a device to facilitate the simpler manufacture of cannulae, particularly cannulae with a rounded trailing end of the cannula aperture. Another subordinate aspect of the object is achieved through the provision of a correspondingly manufactured cannula.

In a first aspect of the invention a method for manufacturing a cannula is provided, which includes providing a hollow tubular segment; and producing a planar cut to create a beveled penetrating entity with a leading penetrating section and a trailing aperture section, wherein the leading penetrating section has a beveled surface that extends rearward from a penetrating tip and has inner lateral edges, and wherein the trailing aperture section has a beveled surface that extends rearward from the beveled surface of the leading penetrating section and has inner lateral edges as well as a rounded trailing edge, wherein both planar cut and the rounding of trailing edge are produced by means of electrochemical machining (ECM) in the presence of an electrolyte.

In a related aspect of the invention a method for manufacturing a cannula is provided, which includes providing a hollow tubular segment; and producing a planar cut to create a beveled penetrating entity with a leading penetrating section and a trailing aperture section, wherein the leading penetrating section has a beveled surface that extends rearward from a penetrating tip and has inner lateral edges, and wherein the trailing aperture section has a beveled surface that extends rearward from the beveled surface of the leading penetrating section and has inner lateral edges as well as a rounded trailing edge, and wherein the penetrating tip of the penetrating entity acquires a sharp tip geometry due to lateral facet cuts, characterized in that the planar cut as well as lateral facet cuts are created using grinding and/or electrical discharge machining before the trailing edge is rounded by means of electrochemical machining (ECM) in the presence of an electrolyte, wherein areas of the penetrating entity that characterize the sharp tip geometry and are not to be subjected to electrochemical machining are shadowed or masked in such a manner that these areas are protected against contact with electrolyte.

In another related aspect of the invention, a device is provided. The device is for manufacturing a cannula from a hollow tubular segment using a planar cut to create a beveled penetrating entity with a leading penetrating section and a trailing aperture section, wherein the leading penetrating section has a beveled surface that extends rearward from a penetrating tip and has inner lateral edges, and wherein the trailing aperture section has a beveled surface that extends rearward from the beveled surface of the leading penetrating section and has inner lateral edges as well as a rounded trailing edge, wherein both planar cut and the rounding of trailing edge are produced by means of electrochemical machining (ECM) in the presence of an electrolyte, and wherein the penetrating tip of the penetrating entity acquires a sharp tip geometry due to lateral facet cuts. The device includes the following components or modules as constituents or functionally dedicated units: a tool electrode for creating planar cut of tubular segment and for rounding trailing edge of the aperture section through electrochemical machining; a tool electrode (6) for creating lateral facet cuts of tubular segment by means of spark erosion, and/or means for grinding; a workpiece holder for accommodating and clamping multiple tubular segments to be processed; and a container for providing the fluids (electrolyte; dielectric material) required for the electrochemical machining and the required electrical discharge machining, if necessary, or means with which the respective fluid can be conveyed to an intended target site.

In another related aspect, another device is provided. The device is for manufacturing a cannula from a hollow tubular segment using a planar cut to create a beveled penetrating entity with a leading penetrating section and a trailing aperture section, wherein the leading penetrating section has a beveled surface that extends rearward from a penetrating tip and has inner lateral edges, and wherein the trailing aperture section has a beveled surface that extends rearward from the beveled surface of the leading penetrating section and has inner lateral edges as well as a rounded trailing edge, wherein the rounding of trailing edge is produced by means of electrochemical machining in the presence of an electrolyte, and wherein the penetrating tip of the penetrating entity acquires a sharp tip geometry due to lateral facet cuts. The device includes the following components or modules as constituents or functionally dedicated units: a tool electrode for creating a planar cut of tubular segment and lateral facet cuts through electrical discharge machining, and/or means for grinding; a tool electrode for rounding trailing edge of the aperture section by means of electrochemical machining; a means for shadowing or masking areas of the penetrating entity that characterize the sharp tip geometry and are not to be subjected to electrochemical machining; a workpiece holder for accommodating and clamping multiple tubular segments to be processed; and a container for providing the fluids (electrolyte; dielectric material) required for the electrochemical machining and the required electrical discharge machining, if necessary, or means with which the respective fluid can be conveyed to an intended target site.

### BRIEF DESCRIPTION OF THE DRAWINGS

FIG. 1 depicts steps for manufacturing a cannula tip from a tubular segment.

FIG. 2, panels (A) and (B) provide schematics illustrating electrochemical machining (ECM) to form a cannula tip 2.

FIG. 3 is a schematic illustrating the selective processing of areas of the cannula.

FIG. 4 depicts the positioning of the electrode 6 during electrochemical removal.

FIG. 5, panel (A) depicts the positioning of the electrode 6 to form a desired geometry. FIG. 5, panel (B) depicts an alternative procedure for simultaneous creation of facent cuts following a planar cut.

FIG. 6, panels (A) and (B) are photographs of a conventional cannula. FIG. 6, panels (C)-(E) are photographs of a cannula formed by ECM.

FIG. 7, panel (A) is a side view of a workpiece holder 18 for tubular segments 1. FIG. 7, panel (B) shows a workpiece holder 18 with clamped tubular segments 1 in a vertical arrangement with a pressure chamber 24. FIG. 7, panel (C) shows a segment of a pressure chamber 24 of panel (B) with a tubular segment 1.

FIG. 8 is a side view of the arrangement of pressure chamber 24, workpiece holder 18, electrode 6 and flushing device 27.

### DETAILED DESCRIPTION

For the purposes of the present invention, cannulae are provided by separating segments of a hollow tube and by using a mostly multi-stage grinding or cutting technique on one end of each of the segments, wherein the separation can be done in a conventional manner by means of cutting processes such as e.g. abrasive friction cutting or sawing, or through electrical discharge machining (spark erosion, sink erosion, wire erosion; also referred to here as EDM).

In the present case, the design of the cannula tip is described as a beveled tip and customarily has three successive cuts, namely the planar cut, which is present in all cannulae and with which a blunt end of a separated tubular segment is given an initial bevel, and, depending upon the subsequent area of application of the finished cannula, a left and a right facet cut or back cut if desired to create the exterior penetrating or cutting edges, wherein the two facet cuts can also be made simultaneously depending upon the technique used for this purpose. For the purposes of the present invention, both the planar cut and the two lateral facet cuts can be provided by means of cutting processes, in particular through the use of grinding or cutting discs, by electrical discharge machining (EDM), in particular wire erosion or sink erosion, through electrochemical machining (also referred to here as ECM), or through the use of a combination of the aforementioned techniques.

Regardless of the specific selection of the technique from the preceding list for creating a cannula tip, the essential aspect of the present invention lies in carrying out the processing (rounding off, rounding) of the trailing end of the cannula aperture, which is also described as the heel and/or eye thereof, electrochemically by means of ECM, which leads to two alternative methods that will be explained in greater detail below.

The abbreviation ECM here stands for electrochemical machining. Like electrochemical polishing, ECM belongs to the group of electrochemical machining methods, although the two methods differ from one another in significant respects. Whereas electrochemical polishing primarily uses acidic electrolytes (e.g. mixtures of sulfuric acid and phosphoric acid), ECM primarily uses neutral electrolytes (saline solutions, e.g. sodium nitrate). The distance between the electrode and the workpiece is very small in the ECM process, usually in the range of 0.05 to 1 mm, whereas in electrochemical polishing, this distance is significantly greater than 1 mm. The current densities in ECM are also usually significantly higher than in electrochemical polishing (A/cm² as compared to mA/cm²). These differences are ultimately due to the different objectives of the two methods. Electrochemical polishing is used for smoothing surfaces, whereas ECM is used for localized removal.

As already stated above, the use of cutting or grinding discs to create the planar cut and/or the two lateral facet cuts produces burrs, which must then conventionally be removed by mechanical means, e.g. through glass bead blasting or fine sand blasting. In addition, the eye must be rounded and the impurities on and/or in the cannula resulting from grinding and blasting must be cleaned off. To smooth the cut services and remove remaining burrs, the cannulae can alternatively or subsequently be subjected to electrochemical polishing (also referred to here as EP).

Alternatively, the planar cut and/or the lateral facet cuts can be produced for the purposes of the present invention by means of EDM, in particular through wire erosion or sink erosion, whereas the eye is subsequently electrochemically processed according to the invention by means of ECM. It is true that this alternative process does not create any burrs that would have to be subsequently removed, e.g. by blasting, and moreover no impurities or obstructions of the cannula would arise through mechanical grinding or cutting processes, and thus along with blasting, the customary electrochemical polishing could also be omitted; nevertheless, the EDM technique is a slow method and suitable equipment is comparatively expensive, for which reason this technique is only suited to a limited extent for inexpensive mass production of cannulae, although it is nevertheless encompassed by the present invention.

Compared with the EDM technique (particularly wire erosion and sink erosion) as a non-contact alternative to mechanical grinding, relatively large quantities of material can be removed from surfaces within a relatively short time using the similarly non-contact ECM process, wherein the quantity to be removed is freely scalable to a limited extent by increasing the electrode surface and current flow, whereas material removal by means of sparks in the EDM technique occurs merely at specific points. Nevertheless, because when using EDM the material is always removed at the point where the distance between the wire and the workpiece is the smallest, rough areas can be effectively smoothed and very precise contours created, which is particularly advantageous when creating sharp edges and tips. By contrast, material removal with electrochemical machining (ECM) occurs in principle where the electrical field lines are the most dense, which is the case particularly in the area of the edges of a cannula tip, whereby specific areas thereof can be rounded in a desired manner.

According to a preferred embodiment, it is therefore proposed to use electrochemical machining (ECM) for the rough, time-intensive material removal of the planar cut and, if desired, to achieve the final forming by means of facet cuts using EDM, wherein during the creation of the planar cut by means of ECM, the rounding of the eye as the trailing end of the cannula aperture also occurs simultaneously. In the case of the preferred combination of ECM and EDM (in this sequence or the reverse) according to the invention, simplified devices can be used and time-intensive reclamping of a plurality of tubular segments to be processed can be omitted, since the workpieces to be processed need to be clamped only once into a workpiece holder, which is a constituent of a device according to the invention or can be incorporated into the different processing stations or modules of such a device as a compatible system component.

According to a preferred embodiment, a suitable device for this embodiment comprises two (for ECM/EDM; EDM/ECM) polarizable tool electrodes, means for contacting and polarizing the cannulae or tubular segments (workpieces) to be processed, and the fluids (electrolyte solution; dielectric material) required for the performance of ECM and EDM, if necessary, or at least functional elements or modules to facilitate the conveyance of the fluids to the intended target site. If EDM is not used (alone), the device alternatively or additionally comprises suitable means for grinding. In alternative methods likewise encompassed by the present invention, in which ECM, although not EDM, is used, a suitable device preferably comprises merely an ECM-compatible tool electrode and only the electrolyte solution and/or means for the conveyance thereof. If the intent is to protect certain areas of the cannula tip completely or to a limited extent against the effect of ECM on the material of the cannula, which can be the case in particular with successive use of EDM and ECM or the use of ECM after the planar cut and/or facet cuts are created alternatively, e.g. through conventional grinding, or with the use of ECM for all cuts (planar cut and facet cuts, if desired), the device further comprises a suitable means with which the areas not to be processed with ECM or to be processed only in an attenuated fashion with ECM can be masked, shadowed, or protected in such a manner that they cannot be contacted by the electrolyte fluid used for processing with ECM, or can be contacted only in an attenuated, i.e. diluted, form.

Depending upon the specific embodiment, the fluid(s) can be provided in one container or, in the case of a combination of both techniques (ECM, EDM), alternatively also in two containers. Alternatively or in addition, one or both fluids can also be provided in the working gap between the workpiece and the tool in another manner, as described in detail below.

For the case preferred according to the invention of creating the planar cut with simultaneous rounding of the eye by means of ECM and the subsequent creation of the final form of the cannula tip by means of bilateral facet cuts through EDM and/or grinding, there is no fear of impairment of the final form and property of the cannula tip because the processing by means of ECM was already completed beforehand and consequently no undesired rounding or blunting can occur in the area of the tip. In this case, the electrolyte can be streamed around or streamed through the given tubular segment end to be processed, without further measures.

For the case proposed alternatively according to the invention of creating the planar cut and the two facet cuts through mechanical grinding/cutting and/or EDM and the then subsequent processing of the eye by means of ECM, it must be ensured that the material removal through ECM is locally restricted, i.e. occurs selectively in the region of the eye, since the desired, previously created property (geometry) and sharpness of the tip and the edges could otherwise be impaired. This applies similarly for the previously presented embodiment of using EDM to create all of the cuts and for the case of a combination of EDM and mechanical grinding/cutting to create the cannula tip, since the electrochemical processing is then carried out subsequently in each case.

With the method of electrochemical machining (ECM), the workpiece (cannula) is polarized as an anode (positive) and the tool electrode as a cathode (negative). In general, the form of the tool cathode is prescribed by the form of the workpiece, for which reason ECM is also generally described as an imaging process, in which no wear occurs on the tool as a result of the process. Depending upon the electrical parameters and the streaming conditions of the electrolyte, a gap (working gap) must be set between the tool and the workpiece (cannula), wherein the width of the gap is between 0.05 and 1 mm. The charge transfer in the working gap is taken over by an electrolyte solution, e.g. an aqueous solution of sodium chloride (NaCl) or preferably sodium nitrate (NaNO₃). The resulting electron stream in the use of ECM produces the desired material removal in that metal ions are released from the cannula. The released metal ions then react with portions of the split electrolyte on the anode (cannula), while the remainder of the electrolyte reacts with water on the cathode (tool electrode), whereby undesirable deposits such as metal hydroxide occur as end products.

As already stated above, according to the invention, electrochemical machining (ECM) in the context of manufacturing cannulas serves in particular to round off the inside of the elliptical eye produced as a result of the planar cut, howsoever created, in the trailing area of the cannula aperture. Nevertheless, because the smallest cannulae have an external diameter of approximately 0.25 mm, it is currently extremely difficult or even impossible to produce according to the aforementioned general teaching an electrode as a tool cathode that is small enough to manifest its electrochemical activity only in the interior of the eye and can be positioned correspondingly precisely at the desired target site. If the electrode is larger or wider than the area to be rounded in the trailing section of the leading, beveled cannula aperture, this can result in undesired material removal outside of the eye and deposits.

For embodiments of the method in which the planar cut and the rounding of the resulting eye by means of ECM have not already been produced, it is therefore proposed according to the invention that the cannulae or areas thereof to be shadowed (masked), such as cannula tips previously created through grinding/cutting and/or erosion, be streamed against from the outside, if necessary, with a non-conductive fluid such as deionized water (DIW), with a fluid that has a sufficiently low conductivity, or with water during their further processing by means of ECM, and to provide the electrolyte stream within the lumen of the cannulae, for which reason a suitable device has means or elements such as e.g. a pressure chamber, with the help of which the electrolyte can preferably be introduced from the end of the tubular segment not being processed into the lumen thereof. Removal therefore occurs only where the concentration of the electrolyte is sufficiently high. This removal preferably occurs on the inside of the eye. As a result of the dilution of the electrolyte outside of the region of the eye, which is caused by the gradual mixing of the electrolyte with the nonconducting fluid, for example, used as a masking means, one advantageously obtains in the area of the lateral cut edges a smooth, continuous transition from areas with removal (rounded off edges) to areas without removal (sharp edges, tip), wherein the area with removal can extend from the region of the eye up to the facet cuts, although it involves at least the trailing edge of the cannula aperture (eye) and directly bilaterally adjacent areas of the edges of the aperture section created by the planar cut. As a result of the stream of fluid on the outer surface of the cannula, the reaction products are diluted and flushed away, for which reason there are also no deposits on or in the cannula. As an alternative to dilution with water, compressed gases (e.g. compressed air) or a fluid (e.g. long-chain fluid alkane such as dodecane) that is not mixable with the electrolyte can be used for complete or partial displacement of the electrolyte in order to mask or shadow areas of the cannula tip not to be processed by means of ECM. For the purposes of an alternatively preferred embodiment, in which the facet cuts are created by means of ECM, the flushing or masking fluid is guided through the cannula or the workpiece while the electrolyte is streamed against or streamed around the cannula or the workpiece.

In general, a shadowing or masking of areas not to be processed can also be accomplished with the alternative or additional temporary use of physical means such as e.g. pads, stamps, and gaskets, or the like made of e.g. elastomers, silicon, rubber, etc., and e.g. the tip geometry can be protected during subsequent treatment with ECM in that the (sharp) tip is inserted into a pad, for example, so that it can no longer be contacted by the electrolyte. For example, the pad can be pressed against the sharp end of the cannula or the workpiece by means of a threaded rod or a pneumatic cylinder in such a manner that the tip with its areas to be protected penetrates into the pad, and thus these areas are protected against contact with the electrolyte. A movement of the cannula or the workpiece through a change in position of the workpiece holder in the direction of the pad or a movement of both objects (pad, cannula) towards each other would similarly be useful. In this process, the pad, the stamp, or the gasket(s) can be attached to the device or to a module thereof, including the workpiece holder, or can be fixedly connected to the electrode. It can be advantageous to insert the cannula into the pad only as far as necessary, so as not to prevent the activity of the ECM electrode and the electrolyte exiting from the cannula. The pad can be preferably designed as a flat disk, or even more preferably as a cylinder, so that by rotating the cylinder accordingly, unpierced areas of the pad can constantly be provided to the cannulae or workpieces during continuous processing. To prevent damage to the pad caused by piercing, if necessary the pad may be preformed in such a way that the cannulae or workpieces can butt against it, although without piercing into the pad.

Against the background of the preceding statements, it is clear that this area-specific masking or shadowing is generally required only in cases or embodiments in which ECM is used after the cannula tip has already received its final form. Because the planar cut, and thus also concomitantly the rounding of the eye, can already be created by means of ECM, a subsequent masking can be omitted, thus making this the most preferable method according to the invention. In exceptional cases, in which e.g. an overly excessive removal in the area of the eye leads to an undesired sharpening of the edge, or in cases in which the facet cuts are created through the use of ECM (see above), a corresponding masking or shadowing or flushing may nevertheless be advisable, particularly at the end of the ECM process.

Insofar as flushing, displacement, masking, or shadowing is referred to in the foregoing, this measure generally relates to the exterior wall of the tubular segment and the electrolyte is provided inside the cannula (within the lumen thereof). Depending upon the desired geometry of the cannula tip to be produced and/or the technique used to create the cuts, however, the flushing, displacement, masking, or shadowing can also affect the interior of the tubular segment or areas of the cannula aperture, in particular the areas in the penetrating section, while the electrolyte streams around the entire exterior or exterior areas of the segment or the cannula.

A cannula manufactured according to the invention with an eye rounded off by means of ECM is not known in the prior art, for which reason the invention also comprises as such a cannula in which the trailing edge (eye) and at least bilateral adjacent areas of the eye (lateral edges created by the planar cut) are blunted or rounded up to any existing facet cuts.

The method according to the invention and the device presented here are therefore well-suited for a simple and cost-effective manufacture of cannulas for medical purposes. This method is simple due to the elimination of the conventionally additional processing steps and the need for multiple clamping and reclamping of cannulae in different devices or in workpiece holders as a constituent or functionally dedicated component of the device.

In general, the method according to the invention can include yet another process step of electrochemical polishing for deburring, sharpening, and/or polishing, wherein it is also preferable in this case that only a correspondingly equipped device is used that, in addition to the previously mentioned components or modules, also comprises a container with an electrode (cathode made of e.g. stainless steel) for accommodating an acid electrolyte (e.g. mixtures of H₂SO₄/H₃PO₄/H₂O).

The invention is explained in greater detail below based upon the figures. Identically acting elements are marked here with the same drawing references where appropriate.

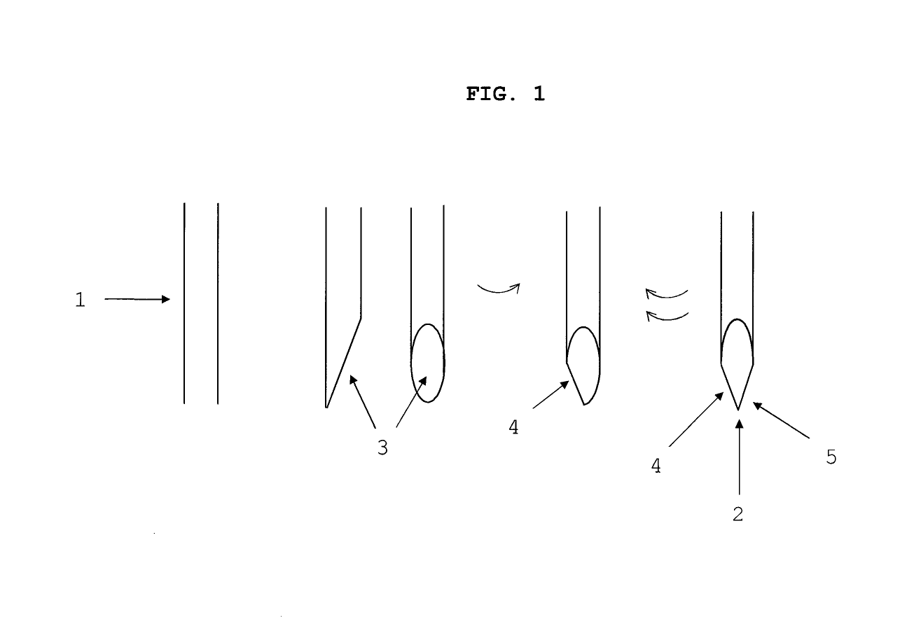

FIG. 1 shows the known steps for manufacturing a cannula tip 2 from a tubular segment 1. To create a beveled tip, planar cut 3 (left in the side view, right in the top view) is made first, followed by the two facet cuts 4 (left) and 5 (right) to be made laterally to the planar cut. To create planar cut 3, tubular segment 1 is in the zero position relative to its longitudinal axis, while it is rotated around its own longitudinal axis, although with an opposing rotational direction, each in a defined angle to create the two facet cuts 4 and 5, respectively, wherein the angles are between 10° and 90° from the zero position and can be selected on a customer-specific basis.

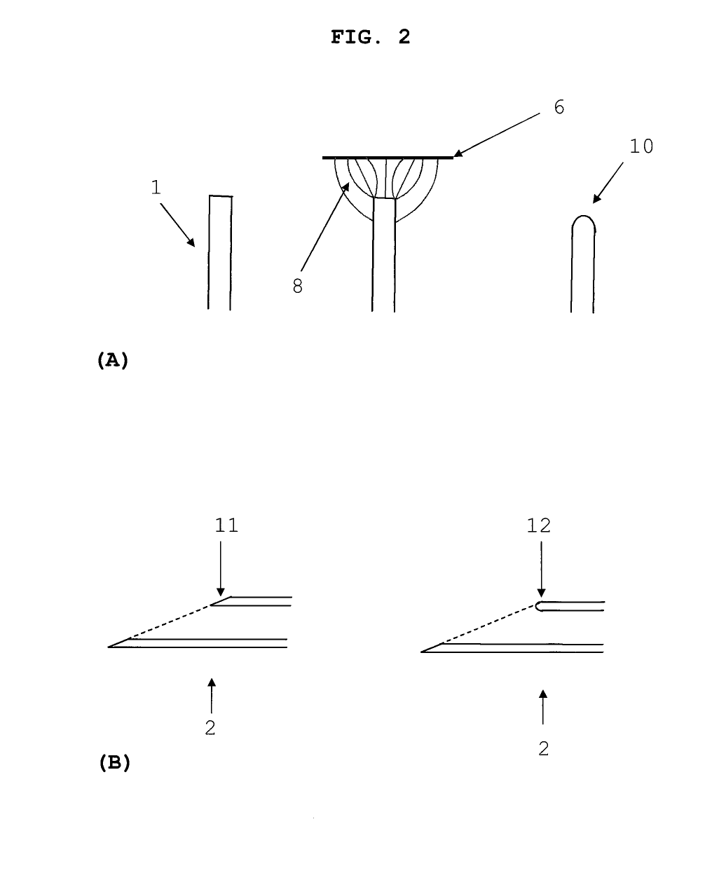

FIG. 2 schematically illustrates the function of electrochemical machining (ECM). A tubular segment 1 represented on the left of panel (A), which as workpiece 8 is polarized as an anode, is electrochemically processed by means of an electrode 6 polarized as a cathode in the presence of an electrolyte 7 (e.g. NaNO₃), whereby a rounded end 10 is created as a result, since the electrical field lines 8 are denser on the edges and tip, and favor the removal of material there.

Panel (B) of FIG. 2 shows the rounding effect produced by ECM in the trailing area of the cannula aperture. The left portion of the figure shows a side view of a cannula, which after the use of EDM and/or conventional grinding possesses merely a planar cut or a finished bevel cut including two facet cuts, wherein the trailing area of the cannula aperture has a sharp eye 11, whereas with the use of ECM, a blunt or rounded eye 12 is obtained. The figure on the right likewise shows that the tip of the cannula, i.e. the projecting end of cannula tip 2 obtained through planar cut or beveled cut, continues to have a sharp form.

In specific applications of the ECM technique, material removal may occur not only in the area of the eye, but also in the area of the (actual) tip as a result of stray electrolysis if an electrolyte bridge is present in this area. Besides the desired rounding of the eye, this can also cause the wall surrounding the eye to be thinned out or roughened and discolored. Because the diameter of the cannulae is very small, it is also difficult to position the electrode (e.g. comb or bar electrode) precisely over only the eye and simultaneously ensure that removal occurs only here, and thus areas not to be processed are preferably masked, shadowed, or protected against removal by other means. Such a masking or shadowing to stop the flow of current and thus protect against undesired removal can create undesired increments in the transition area between unprotected and protected areas, however. Moreover, an exact positioning of a mask or covering is also difficult with small cannulae.

Should this problem occur, it can be solved in that the areas not to be processed, i.e. to be protected, are streamed against or flushed with e.g. water in such a manner that these areas cannot be contacted by the electrolyte or can be contacted only by diluted electrolyte.

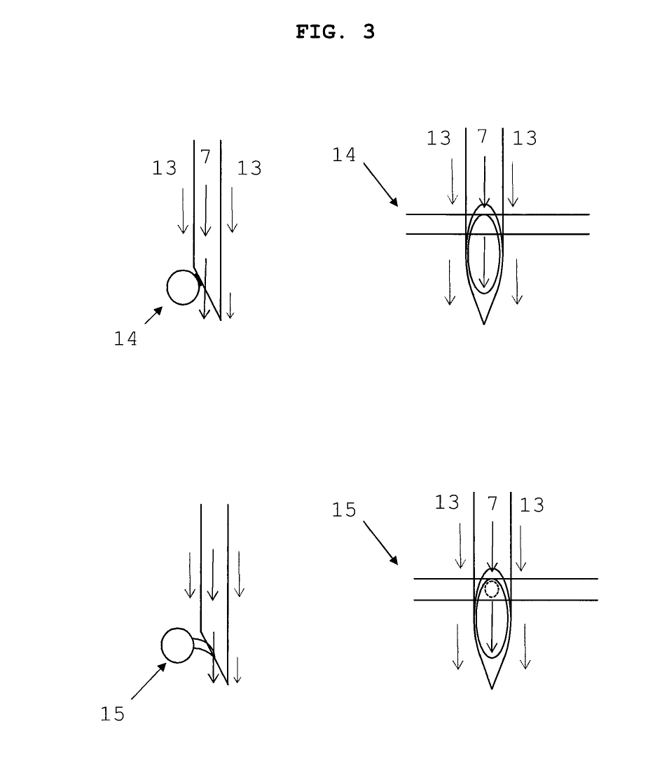

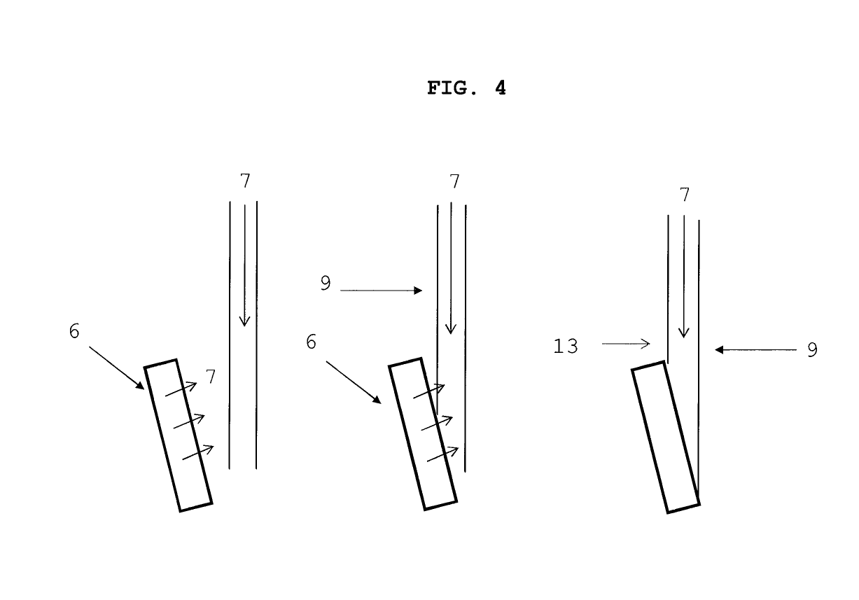

As shown in FIG. 3, the areas not to be processed in this case are streamed against or flushed with water 13, while the cannula is charged or flowed through with electrolyte 7 from the inside e.g. through a pressure chamber. The electrode, such as e.g. a bar electrode 14, is positioned before the eye, and the electrolyte exiting at the eye from the trailing area of the cannula aperture forms an electrolyte bridge between the eye and electrode 14. The further one moves from the eye in the direction of the tip, the more electrolyte 7 is diluted by the water flushing, whereby a smooth, continuous transition between areas with removal and areas without removal is obtained. Electrolyte 7 is therefore (largely) undiluted in the area of the eye, for which reason the removal is greatest here, which is desired for the rounding of the eye. The longitudinal extent of these areas, which are to be treated differently, can be influenced by setting the ratio between electrolyte flow and water flow, wherein it must be ensured in any case that the tip geometry of the penetrating section obtained through the cuts remains largely intact. This is achieved in that these areas are not contacted at all or only in a very much diluted fashion by electrolyte 7 such that no noteworthy removal can occur here. Instead of bar electrode 14 shown here, alternatively a comb electrode 15 that extends into the eye can also be used with larger tube diameters.

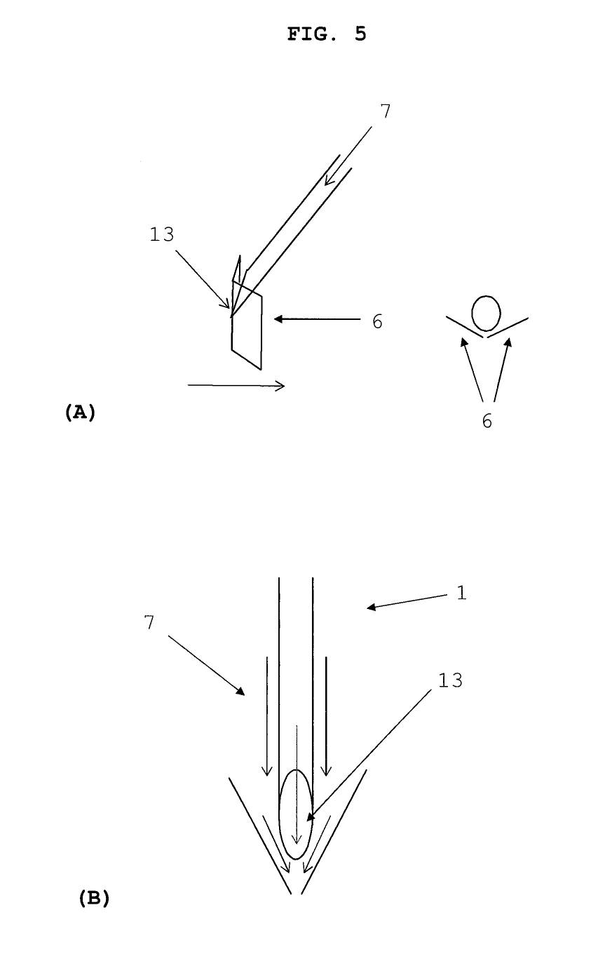

As already shown, the material removal as well as the cut length of the planar cut as the first step to the manufacture of a cannula tip is the largest, for which reason the ECM technique is preferred over the EDM technique for this purpose. To obtain a rapid and even removal, tubular segment 1 pursuant to the illustration in FIG. 4 should be sufficiently streamed against in the area to be removed by a possibly undiluted electrolyte 7, for which the interior (lumen) of tubular segment 1 is charged or flowed through with electrolyte 7. Alternatively or in addition, an electrode 6 can be used, which is designed so that electrolyte 7 can flow through it and which has boreholes through which electrolyte 7 can be conveyed to workpiece 9. Also if ECM is used to create the planar cut with simultaneous rounding off of the eye, it can be necessary to flush the tubular segment in specific areas with e.g. water in order to avoid undesired or excessive removal e.g. around the eye. To reduce the dilution of the electrolyte as desired through the water flushing, however, it can be useful to activate the water flushing only in parts, such as at the end of the ECM process, for example. As shown in FIG. 4, the gap between electrode 6 and tubular segment 1 or workpiece 9 during the electrochemical removal is kept largely constant by repositioning electrode 6. Because the edges are in principle rounded by means of ECM (cf. FIG. 2), a rounding of the eye occurs simultaneously with the creation of the planar cut. The creation of the facet cuts occurs subsequently, preferably by means of electrical discharge machining (EDM).

As already stated, all cuts of the beveled tip (planar cut, facet cuts) can be created by means of ECM. For this purpose, after the previously described ECM planar cut, one or two additional ECM steps occur to create the facet cuts. Because the electrochemical machining in principle rounds off edges and tips, however, all edges, in particular also the tip created by the planar cut, are round if contacted with electrolyte. To obtain a “pointed” tip with sharp edges, it can also be desired for the purposes of this embodiment to provide a corresponding flushing with water, which can occur with reference to FIG. 5, for example, in that the tip is streamed against or flushed with water 13, thus preventing removal on the tip. If the tubular segments are clamped at a certain lateral distance from one another in the workpiece holder of the device, it is possible to create the facets on both sides simultaneously in one step, wherein the spatial configuration of electrode 6 must then be made in accordance with the desired tip geometry, as shown in panel (A) of FIG. 5. Alternatively or in addition to rinsing, masking, or shadowing, the desired sharpness of the tip and the edges in the penetrating section can be created by means of electrochemical polishing (EP). An alternative procedure for simultaneous creation of both facet cuts following a previously made planar cut is shown in panel (B) of FIG. 5. According to this embodiment, the cannula is flushed from inside with e.g. water, while electrolyte is streamed around it from the outside. As soon as the water stream fed into the lumen of the cannula exits from the cannula aperture in the direction of the tip, it is increasingly mixed with electrolyte in the direction of the tip and from outside to inside, whereby its concentration is strongest in the area desired for creation of the facet cuts, which is positively supported through the use of an angled electrode. The area of pure water in the fluid stream on the eye is thus as broad as the tube inner diameter, and diminishes successively and evenly towards the tip. Due to a suitable ratio between the water stream and electrolyte stream, an increasing concentration with respect to electrolyte can be achieved along the penetrating section of the cannula, whereby the creation of the desired tip geometry is supported.

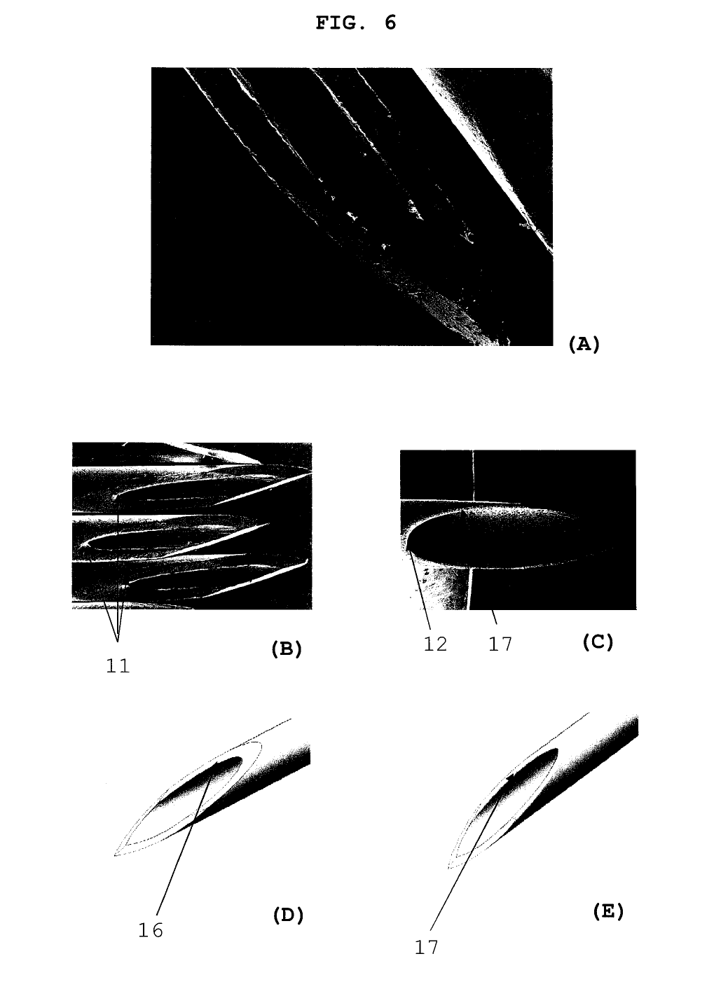

In FIG. 6, the properties of a cannula tip according to the invention, which are superior to those of conventionally manufactured cannula tips, are shown in comparison. panel (A) of FIG. 6 is an image of a conventionally created cannula tip with residual burrs clearly present, whereas the image pursuant to panel (B) also shows the continued elliptical (sharp) form of the trailing and clearly inwardly curved area of the cannula aperture (sharp eye 11) of a conventional standard cannula. The image pursuant to panel (C), by contrast, shows the area of an eye 12 rounded off according to the invention by means of ECM. As also evident in panels (D) and (E) of FIG. 6, the particular leading penetrating section with its tip and it sharp edges (tip geometry) is equally formed in both versions. The differences in the trailing aperture section are that much more pronounced, however. Whereas this aperture section created in the conventional manner (EDM and/or grinding) has a flat design and has both sharp inner edges and a sharp trailing edge (eye) (cf. FIG. 6: panel (D)), the aperture section processed by means of ECM has both a rounded or blunted trailing edge (rounded eye) as well as rounded or blunt inner edges, which involve the areas directly adjacent to the eye or even extend, as shown, up to the facets. Thus the inner edges of the entire aperture section of a standard cannula continue to be sharp-edged, whereas these planar cut edges 17 in a cannula tip according to the invention are likewise rounded pursuant to the image in panel (E), wherein a flat design of the aperture section, at least in certain areas, does not contradict the teaching according to the invention, insofar as at least the trailing edge (eye) is rounded without the use of mechanical means such as e.g. brushes, which leave grooves or grinding marks, etc. in the area in question.

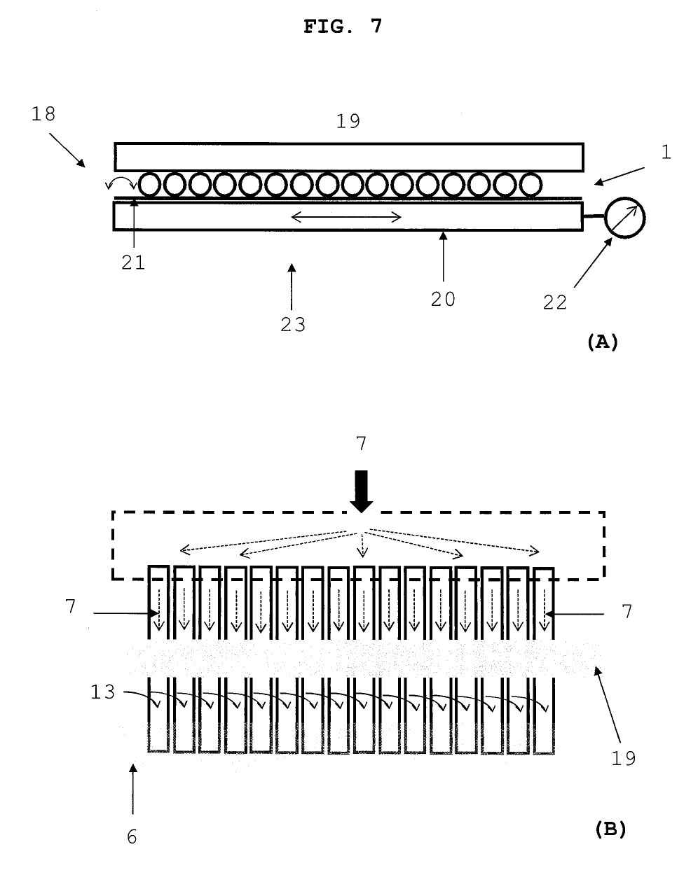

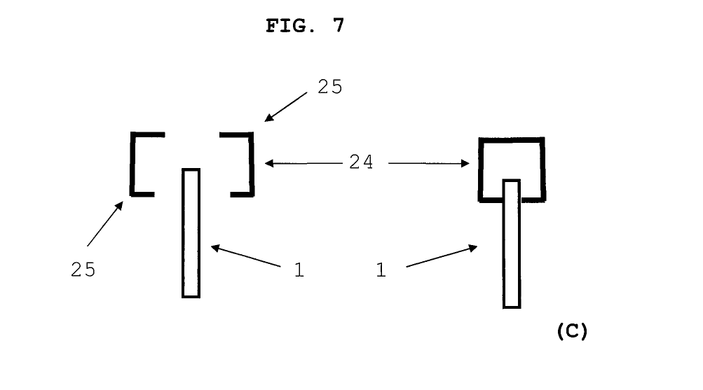

FIG. 7, panel (A) shows a side view of a workpiece holder 18 for holding, and rotating if necessary, and contacting tubular segments 1 and for the transfer of workpieces between individual processing stations or modules of a device. Tubular segments 1 are clamped between two bars 19 and 20, wherein one of these bars, designated here as displaceable bar 20, can be preferably displaced in opposite directions, vertical to the workpieces, whereby the workpieces clamped in zero position relative to their longitudinal axis can be rotated to the left or right after creation of the planar cut in order to create the two facet cuts. To balance out tolerances of tubular segments 1 and increase the friction between displaceable bar 20 and the workpieces, this is preferably coated with a polymer 21. The displacement of bar 20 occurs by means of a pneumatic or electrical actuator (not shown) and is monitored by a measuring device 22. Contact bar 19 facing displaceable bar 20 consists of a conductive material or has at least a conductive coating, and in addition to holding and if necessary rotating tubular segments 1, serves to contact tubular segments 1 or workpieces for electrochemical machining (ECM) and electrical discharge machining (EDM) that is provided if necessary. To ensure a secure contact and rotation of tubular segments 1 or workpieces, the bars 19 and 20 must be pressed together equally with a defined contact pressure, which can be achieved, for example, with the help of springs or pneumatically. To achieve a sufficiently precise positioning of workpiece holder 18 in the individual processing stations of the device, workpiece holder 18 possesses at least one positioning element (not shown), for example in the form of a pin or rod. In the particular stations or processing modules of the device are receiving mechanisms that are compatible with the positioning elements, such as e.g. a zero potential mechanism or prism. Here the arrangement of the workpiece holder in a station or in a module of the device can be horizontal to vertical, depending upon the property of the said station or module. It is therefore possible to process tubular segments 1 or workpieces in different processing stations or modules (e.g. ECM; EDM; EP) without the need to re-clamp tubular segments 1 or workpieces between the stations.

FIG. 7, panel (B) shows a workpiece holder with clamped tubular segments in a vertical arrangement with a pressure chamber 24, with which electrolyte 7 can be introduced into the lumen of the tubular segments and, if necessary, a flushing, masking, or shadowing of exterior areas of the tubular segments not to be processed above electrode 6, which if necessary can also be charged with a medium stream, can be undertaken with water 13. In the event that ECM is used according to the invention to create the planar cut with simultaneous rounding of the resulting eye, the tubular segments are preferably streamed through or against with electrolyte both from inside and through the electrode from outside, while they can be streamed against or flushed with water if desired above the eye, wherein the water preferably streams in a laminar flow along the workpiece for this purpose. In this manner, it can be ensured that a workpiece holder bearing tubular segments or workpieces does not need to be submersed into a medium.

FIG. 7, panel (C) shows a segment of such a pressure chamber or pressure chamber 24 pursuant to Panel B with a tubular segment 1. It consists of two half-shells 25 with an elastomer gasket on the sealing surfaces, wherein the half-shells 25 are opened (left) for changing a workpiece holder and closed (right) during processing. Pressure chamber 24 is preferably a constituent of each ECM processing station, but can also be provided as a module, which is transferred together with the workpiece holder between the different stations or modules of the device, and can be charged with the desired fluid from the compatible accommodation means present there.

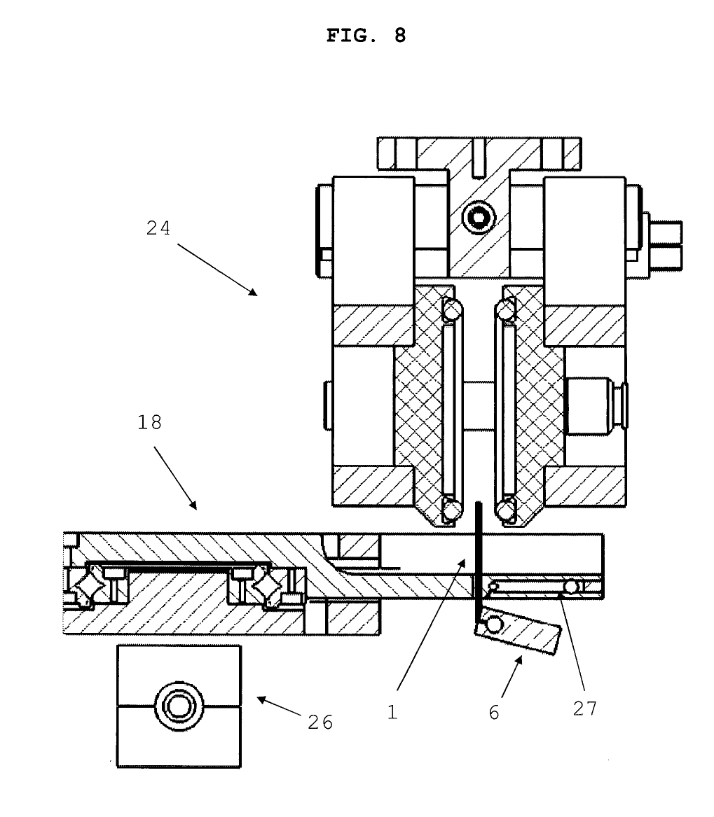

FIG. 8 shows a side view of the arrangement of pressure chamber 24, workpiece holder 18, electrode 6, and flushing device 27. For the purposes of the application shown here, the electrolyte is streamed through tubular segment 1 or workpiece 9 through pressure chamber 24 from the inside, while water is flushed through a flushing nozzle 27 above the eye from the outside, wherein the water should stream in a laminar flow to flush the area to be protected against removal and deposits. Care must be taken, if necessary, however, to ensure that the electrolyte is not diluted, or at least not too strongly diluted, by the water flushing at its designated target site. The scope of the flushing therefore depends upon the specific requirements for the areas to be processed and, if necessary, any areas to be protected, and can be selectively restricted, e.g. to specific areas of the cannula aperture, or applied to the entire cannula tip. Below workpiece holder 18, a drive for rotation 26 of tubular segments 1 within workpiece holder 18 is shown.

For electrical discharge machining (EDM), a conventional wire erosion or sink erosion machine can be used, wherein a corresponding module can also be a constituent of the device or functionally dedicated to it.

The machine or the module is preferably equipped with a clamping device for optional horizontal or vertical accommodation of the workpiece holder and optionally with a previously described pressure chamber for flushing the tubular segments or workpieces from the inside.

### DRAWING REFERENCE LIST

1 Tubular segment, tubular segments
2 Cannula tip, penetrating tip
3 Planar cut
4 First facet cut
5 Second facet cut
6 Electrode (cathode), tool electrode
7 Electrolyte
8 Field lines
9 Workpiece (anode)
10 Rounded end, rounded trailing edge, rounding of the trailing edge
11 Sharp end/eye
12 Rounded eye, rounded trailing edge, rounding of the trailing edge
13 Water, flushing fluid
14 Bar electrode, electrode, tool electrode
15 Comb electrode, electrode, tool electrode
16 Surface of the planar cut
17 Rounded edges of the planar cut
18 Workpiece holder
19 Contact bar, bar
20 Displaceable bar, bar
21 Polymer coating
22 Measuring device
23 Defined contact pressure
24 Pressure chamber
25 Half-shell
26 Drive for rotation
27 Flushing device, flushing nozzle

1. A method for manufacturing a cannula, comprising:
(a) providing a hollow tubular segment (1); and
(b) producing a planar cut (3) to create a beveled penetrating entity with a leading penetrating section and a trailing aperture section, wherein the leading penetrating section has a beveled surface that extends rearward from a penetrating tip and has inner lateral edges, and wherein the trailing aperture section has a beveled surface that extends rearward from the beveled surface of the leading penetrating section and has inner lateral edges as well as a rounded trailing edge (10, 12), wherein both planar cut (3) and the rounding of trailing edge (10, 12) are produced by means of electrochemical machining (ECM) in the presence of an electrolyte (7).

2. The method according to claim 1, wherein the electrolyte (7) is conducted through tubular segment (1) during the electrochemical machining.

3. A method according to claim 1, wherein the penetrating tip of the penetrating entity acquires a sharp tip geometry due to lateral facet cuts (4, 5), wherein facet cuts (4, 5) are created by grinding and/or electrical discharge machining.

4. A method for manufacturing a cannula, comprising:
(a) providing a hollow tubular segment (1); and
(b) producing a planar cut (3) to create a beveled penetrating entity with a leading penetrating section and a trailing aperture section, wherein the leading penetrating section has a beveled surface that extends rearward from a penetrating tip and has inner lateral edges, and wherein the trailing aperture section has a beveled surface that extends rearward from the beveled surface of the leading penetrating section and has inner lateral edges as well as a rounded trailing edge (10, 12), and wherein the penetrating tip of the penetrating entity acquires a sharp tip geometry due to lateral facet cuts (4, 5), characterized in that the planar cut (3) as well as lateral facet cuts (4, 5) are created using grinding and/or electrical discharge machining before the trailing edge (10, 12) is rounded by means of electrochemical machining (ECM) in the presence of an electrolyte (7), wherein areas of the penetrating entity that characterize the sharp tip geometry and are not to be subjected to electrochemical machining are shadowed or masked in such a manner that these areas are protected against contact with electrolyte (7).

5. A method according to claim 4, wherein the shadowing or masking occurs optionally, in that the areas are streamed against or flushed with a fluid selected from the group consisting of deionized water, water, and a fluid that cannot be mixed with electrolyte (7), and/or by displacing electrolyte (7) in the areas by streaming a compressed gas against these areas.

6. A device for manufacturing a cannula from a hollow tubular segment (1) using a planar cut (3) to create a beveled penetrating entity with a leading penetrating section and a trailing aperture section, wherein the leading penetrating section has a beveled surface that extends rearward from a penetrating tip and has inner lateral edges, and wherein the trailing aperture section has a beveled surface that extends rearward from the beveled surface of the leading penetrating section and has inner lateral edges as well as a rounded trailing edge (10, 12), wherein both planar cut (3) and the rounding of trailing edge (10, 12) are produced by means of electrochemical machining (ECM) in the presence of an electrolyte (7), and wherein the penetrating tip of the penetrating entity acquires a sharp tip geometry due to lateral facet cuts (4, 5), wherein the device comprises the following components or modules as constituents or functionally dedicated units:
(a) tool electrode (6) for creating planar cut (3) of tubular segment (1) and for rounding trailing edge (10, 12) of the aperture section through electrochemical machining;
(b) tool electrode (6) for creating lateral facet cuts (4, 5) of tubular segment (1) by means of spark erosion, and/or means for grinding;
(c) workpiece holder (18) for accommodating and clamping multiple tubular segments (1) to be processed; and
(d) container for providing the fluids (electrolyte; dielectric material) required for the electrochemical machining and the required electrical discharge machining, if necessary, or means with which the respective fluid can be conveyed to an intended target site.

7. A device for manufacturing a cannula from a hollow tubular segment (1) using a planar cut (3) to create a beveled penetrating entity with a leading penetrating section and a trailing aperture section, wherein the leading penetrating section has a beveled surface that extends rearward from a penetrating tip and has inner lateral edges, and wherein the trailing aperture section has a beveled surface that extends rearward from the beveled surface of the leading penetrating section and has inner lateral edges as well as a rounded trailing edge (10, 12), wherein the rounding of trailing edge (10, 12) is produced by means of electrochemical machining (ECM) in the presence of an electrolyte (7), and wherein the penetrating tip of the penetrating entity acquires a sharp tip geometry due to lateral facet cuts (4, 5), wherein the device comprises the following components or modules as constituents or functionally dedicated units:
(a) tool electrode (6) for creating planar cut (3) of tubular segment (1) and lateral facet cuts (4, 5) through electrical discharge machining, and/or means for grinding;
(b) tool electrode (6) for rounding trailing edge (10, 12) of the aperture section by means of electrochemical machining;
(c) means for shadowing or masking areas of the penetrating entity that characterize the sharp tip geometry and are not to be subjected to electrochemical machining;
(d) workpiece holder (18) for accommodating and clamping multiple tubular segments (1) to be processed; and
(e) container for providing the fluids (electrolyte; dielectric material) required for the electrochemical machining and the required electrical discharge machining, if necessary, or means with which the respective fluid can be conveyed to an intended target site.

8. A device according to claim 7, wherein the means for shadowing or masking is a fluid selected from the group consisting of deionized water, water, and a fluid that cannot be mixed with electrolyte (7), or a compressed gas.
